(19) United States
(12) Patent Application Publication
(10) Pub. No.: US 2023/0059472 A1
(43) Pub. Date: Feb. 23, 2023

(54) AIR CLEANING APPARATUS AND METHODS OF PROVIDING AND USING THE SAME

(71) Applicant: PURO Lighting, LLC, Lakewood, CO (US)

(72) Inventors: Adrian Armijo, Arvada, CO (US); Alexandre Wamain, Denver, CO (US); Laura Callahan, Denver, CO (US)

(73) Assignee: PURO Lighting, LLC, Lakewood, CO (US)

(21) Appl. No.: 17/891,006

(22) Filed: Aug. 18, 2022

Related U.S. Application Data
(60) Provisional application No. 63/235,079, filed on Aug. 19, 2021.

(51) Int. Cl. A61L 9/20; B01D 53/86; B01D 53/00

(52) U.S. Cl. CPC A61L 9/205; B01D 53/8668; B01D 53/007; B01D 2257/708; B01D 2255/802; B01D 2259/804; A61L 2209/12

(57) ABSTRACT

An air cleaning apparatus including a housing that includes one or more intake vents and one or more exhaust vents. The air cleaning apparatus also can include an air chamber within the housing. The air cleaning apparatus additionally can include one or more disinfecting lights directed toward the air chamber. The air cleaning apparatus further can include a fan operable to perform moving air from outside the housing in a first direction through the one or more intake vents and into the air chamber to be exposed to the one or more disinfecting lights, and, after the air is exposed within the air chamber to the one or more disinfecting lights, moving the air from of the air chamber through the one or more exhaust vents in one or more second directions that are other than approximately parallel to the first direction. The air cleaning apparatus additionally can include a vortex reflector positioned adjacent to the air chamber and across from the one or more disinfecting lights. The air cleaning apparatus further can include vortex-inducing walls within the air chamber. Other embodiments are described.

### CROSS-REFERENCE TO RELATED APPLICATION

This application claims the benefit of U.S. Provisional Application No. 63/235,079, filed Aug. 19, 2021. U.S. Provisional Application No. 63/235,079 is incorporated herein by reference in its entirety

### TECHNICAL FIELD

This disclosure relates generally to air cleaning apparatuses.

### BACKGROUND

Pathogens inside enclosed spaces, such as buildings or transit vehicles, often flow through the air significant distances toward air intakes of heating, ventilation, and air conditional (HVAC) units. For example, if a bus driver or an individual seated near the front of a bus is ill or coughs, those pathogens typically flow toward the rear of the bus, where the HVAC unit is located. Others in the bus along that path risk exposure to such pathogens.

### BRIEF DESCRIPTION OF THE DRAWINGS

To facilitate further description of the embodiments, the following drawings are provided in which:

FIG. 1 illustrates a shell-side perspective view of an air cleaning apparatus, according to an embodiment;

FIG. 2 illustrates a back plate-side perspective view of the air cleaning apparatus of FIG. 1;

FIG. 3 illustrates an internal plan view of the air cleaning apparatus of FIG. 1, with the back plate removed to show internal components of the air cleaning apparatus;

FIG. 4 illustrates a cross-sectional view, along cross-section line 4-4 in FIG. 2, of the air cleaning apparatus of FIG. 1;

FIG. 5 shows a computational flow dynamics air flow diagram for the air cleaning apparatus of FIG. 1;

FIG. 6 shows an exploded view of the air cleaning apparatus of FIG. 1;

FIG. 7 illustrates a transit bus, according to another embodiment;

FIG. 8 illustrates a cross-sectional view, along cross-section line 8-8 in FIG. 7, of the transit bus of FIG. 7;

FIG. 9 illustrates a shell-side perspective view of an air cleaning apparatus with a top hat, according to another embodiment;

FIG. 10 illustrates a hat-side perspective view of the air cleaning apparatus of FIG. 9;

FIG. 11 illustrates a cross-sectional view, along cross-section line 11-11 in FIG. 10, of the air cleaning apparatus of FIG. 9;

FIG. 12 shows an exploded view of the air cleaning apparatus of FIG. 9;

FIG. 13 illustrates an LED board, according to another embodiment;

FIG. 14 illustrates a cross-sectional view of an air cleaning apparatus with a mercury vapor lamp, according to another embodiment;

FIG. 15 shows an exploded view of the air cleaning apparatus of FIG. 14;

FIG. 16 illustrates a flow chart for a method 1600 of providing an air cleaning apparatus, according to another embodiment; and

FIG. 17 illustrates a flow chart for a method 1700 of cleaning air, according to another embodiment.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “approximately” can, in some embodiments, mean within plus or minus thirty percent of the stated value, or within plus or minus thirty degrees of the stated direction, as applicable. In other embodiments, “approximately” can mean within plus or minus twenty percent of the stated value, or within plus or minus twenty degrees of the stated direction, as applicable. In other embodiments, “approximately” can mean within plus or minus ten percent of the stated value, or within plus or minus ten degrees of the stated direction, as applicable. In other embodiments, “approximately” can mean within plus or minus five percent of the stated value, or within plus or minus five degrees of the stated direction, as applicable. In further embodiments, “approximately” can mean within plus or minus three percent of the stated value, or within plus or minus three degrees of the stated direction, as applicable. In yet other embodiments, “approximately” can mean within plus or minus one percent of the stated value, or within plus or minus three degrees of the stated direction, as applicable.

### DESCRIPTION OF EXAMPLES OF EMBODIMENTS

Various embodiments include an air cleaning apparatus. The air cleaning apparatus can include a housing that includes one or more intake vents and one or more exhaust vents. The air cleaning apparatus also can include an air chamber within the housing. The air cleaning apparatus additionally can include one or more disinfecting lights directed toward the air chamber. The air cleaning apparatus further can include a fan operable to perform moving air from outside the housing in a first direction through the one or more intake vents and into the air chamber to be exposed to the one or more disinfecting lights, and, after the air is exposed within the air chamber to the one or more disinfecting lights, moving the air from of the air chamber through the one or more exhaust vents in one or more second directions that are other than approximately parallel to the first direction. The air cleaning apparatus additionally can include a vortex reflector positioned adjacent to the air chamber and across from the one or more disinfecting lights. The air cleaning apparatus further can include vortex-inducing walls within the air chamber.

A number of embodiments include a method of providing an air cleaning apparatus. The method can include providing a housing that includes one or more intake vents and one or more exhaust vents. The method also can include providing an air chamber within the housing. The method additionally can include providing one or more disinfecting lights directed toward the air chamber. The method further can include providing a fan operable to perform moving air from outside the housing in a first direction through the one or more intake vents and into the air chamber to be exposed to the one or more disinfecting lights, and, after the air is exposed within the air chamber to the one or more disinfecting lights, moving the air from of the air chamber through the one or more exhaust vents in one or more second directions that are other than approximately parallel to the first direction. The method additionally can include providing a vortex reflector positioned adjacent to the air chamber and across from the one or more disinfecting lights. The method further can include providing vortex-inducing walls within the air chamber.

Further embodiments include a method of cleaning air. The method can include receiving air from outside a housing of an air cleaning apparatus in a first direction through one or more intake vents of the housing and into an air chamber of the air cleaning apparatus to be exposed to one or more disinfecting lights of the air cleaning apparatus that are directed toward the air chamber. The method also can include, after the air is exposed within the air chamber to the one or more disinfecting lights, exhausting the air from of the air chamber through one or more exhaust vents of the housing in one or more second directions that are other than approximately parallel to the first direction. The air cleaning apparatus can include a vortex reflector positioned adjacent to the air chamber and across from the one or more disinfecting lights. The air cleaning apparatus also can include vortex-inducing walls within the air chamber.

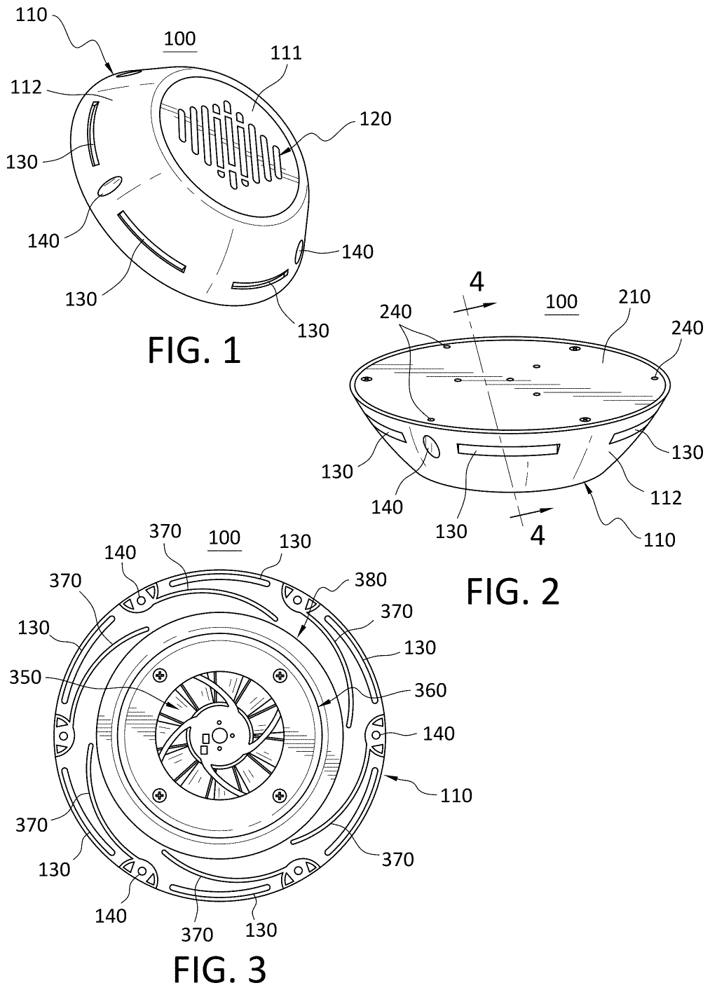

Turning to the drawings, FIG. 1 illustrates a shell-side perspective view of an exemplary embodiment of an air cleaning apparatus 100. FIG. 2 illustrates a back plate-side perspective view of air cleaning apparatus 100. Air cleaning apparatus 100 is merely exemplary, and embodiments of the air cleaning apparatus are not limited to the embodiments presented herein. The air cleaning apparatus can be employed in many different embodiments or examples not specifically depicted or described herein. In many embodiments, air cleaning apparatus 100 can be configured to locally disinfect the upper air in transit vehicles and/or other suitable environments.

As shown in FIGS. 1 and 2, air cleaning apparatus 100 can include a housing, which in some embodiments can include a shell 110 and/or a back plate 210. In a number of embodiments, shell 110 can include a central surface 111 and a side surface 112. In many embodiments, such as shown in FIGS. 1 and 2, central surface 111 and side surface 112 can be shaped as a frustum of a cone, with central surface 111 being substantially flat and side surface 112 being rounded and frustoconical. In other embodiments, shell 110 can have other suitable form factors. In several embodiments, shell 110 can include intake vents 120, such as on central surface 111, and exhaust vents 130, such as around side surface 112 near the outer edge of shell 110. The air flowing into intake vents 120 can flow in a direction approximately perpendicular to central surface 111, and can flow out of exhaust vents 130 in a direction approximately parallel to central surface 111, such that the air exiting exhaust vents 130 is blown away from intake vents 120 and in a direction approximately perpendicular from intake vents 120, which can limit recirculation of air directly from exhaust vents 130 to intake vents 120. In a number of embodiments, shell 110 also can include screw holes 140, which can be paired with screw holes 240 in back plate 210, and which can be used with screws to mount air cleaning apparatus 100 to a surface, such as the ceiling of a transit vehicle.

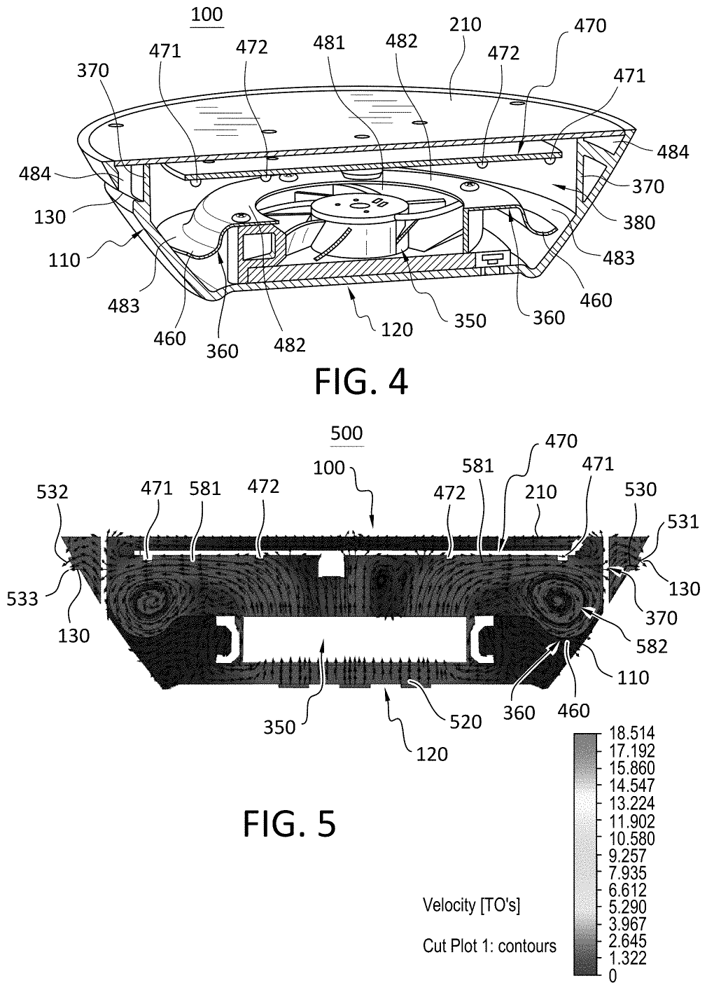

FIG. 3 illustrates an internal plan view of air cleaning apparatus 100, with back plate 210 removed to show internal components of air cleaning apparatus 100. FIG. 4 illustrates a cross-sectional view, along cross-section line 4-4 in FIG. 2, of air cleaning apparatus 100. In a number of embodiments, air cleaning apparatus 100 can include a fan 350, a light emitting diode (LED) board 470, an air chamber 380, a vortex reflector 360, and/or vortex-inducing walls 370. Fan 350 can pull air into air cleaning apparatus 100 through intake vents 120, push the air through air chamber 280, and push the air out of air cleaning apparatus 100 through exhaust vents 130. LED board can be attached to back plate 210, and can include LEDs, such as LEDs 471 and 472, and/or electronics to control the LEDs and/or other components of air cleaning apparatus 100. In many embodiments, the LEDs can be arranged in concentric circles, such as LEDs 471 in an outer concentric circle and LEDs 472 in an inner concentric circle. In many embodiments, the LEDs can produce ultraviolet (UV) light, such as one or more of UV-A (ultraviolet A, approximately 315 to 399 nanometer (nm) wavelength), UV-B (ultraviolet B, approximately 280 to 315 nanometer wavelength) LED, and/or UV-C (ultraviolet C, approximately 100 to 280 nanometer wavelength). UV-C is particularly effective at disinfecting the air from pathogens.

In many embodiments, vortex reflector 360 can be located around fan 350 and can extend radially outward toward the sides (e.g., 112) of air cleaning apparatus 100. In a number of embodiments, such as shown in FIG. 4, vortex reflector 360 can curve away from LED board 470 as vortex reflector 360 extends radially outward and them back toward LED board 470 to create a concave region 460. In many embodiments, air chamber 380 can include an intake region 481, a radial region 482, a vortex region 483, and an exhaust region 484. When fan 350 blows air from outside air cleaning apparatus 100 through intake vents 120 and into air chamber 380, the air can begin at intake region 481, after which it is pushed radially outward in radial region 482 toward a vortex region 483, after which the air enters exhaust region 484 before exiting through exhaust vents 130. Fan 350 can move air through air chamber 380 to be disinfected and at the same time cool LED board 470 and the LEDs and electronics thereon while air cleaning apparatus 100 is operating. This active cooling can allow a larger current to be supplied to one or more LEDs, which in turn output more UV energy to increase disinfection of the air. The cooling can beneficially increase the lifespan of LED board 470.

In many embodiments, vortex-inducing walls 370 can substantially cover exhaust vents 130 to prevent air from blowing directly radially out of air chamber 380, and, in conjunction with concave region 460 of vortex reflector 360, can cause some of the air in air chamber 380 to create a vortex in vortex region 483 of air chamber 380, which can increase the dwell time of air inside air chamber 380 to increase disinfecting effectiveness. Vortex-inducing walls 370 can be attached to and/or formed as part of shell 110, and can curve radially inward to redirect air in air chamber 380 in a clockwise or counterclockwise direction around a perimeter of the air cleaning apparatus before escaping through exhaust vents 130. In this way, air can be further delayed within the device, which then can provide the air with more exposure to the UV light.

FIG. 5 shows a computational flow dynamics (CFD) air flow diagram 500 for air cleaning apparatus 100. Vortex-inducing walls and/or the shape of vortex reflector 360, such as at concave region 460, can force air into one or more vortexes. For example, air can be pulled through intake vents 120 into air cleaning apparatus 100 by fan 350 with the air moving in a direction 520 (e.g., in the direction from central surface 111 (FIGS. 1-2) toward back plate 210 (FIG. 2)). The air enters air chamber 380 (FIGS. 3-4) at intake region 481 (FIG. 4), and the air is then pushed outward through radial region 482 (FIG. 4) in a direction 581 (e.g., radially outward from intake region 481 (FIG. 4)). As the air nears vortex-inducing walls 370 in vortex region 483 (FIG. 4), the air begins to circulated in a vortex 582, such as by circulating away from LED board 470 near vortex-inducing walls 370, back toward the center near concave region 460, back toward LED board 470, and then back toward vortex-inducing walls 370. These vortexes can be created at each of vortex-inducing walls 370, which can extend the time air dwells in air chamber 380 being disinfected.

In many embodiments, an additional vortex can be simultaneously created by air cleaning apparatus 100 to further extend time air spends in the air cleaning apparatus. For example, vortex-inducing walls 370 can direct at least a portion of the air in a clockwise (or counterclockwise) direction within vortex region 483 (FIG. 4), as the air moves toward exhaust region 484 (FIG. 4), and then out exhaust vent 130. In this way, vortex-inducing walls 370 can (1) initiate a vortex created by movement of the air around vortex reflector 360 and (2) initiate a vortex circling around the perimeter of air cleaning apparatus 100 inside of vortex-inducing walls 370. In many embodiments, one or more LEDs (e.g., 471-472) can be placed directly over a vortex, such as in one, two, or more concentric circles of LEDs over vortex region 483 (FIG. 4). In this way, UV exposure of air taken into air cleaning apparatus 100 can be maximized. Eventually, the air moves into exhaust region 484 (FIG. 4), and is exhausted out exhaust vents 130. The air exhausting out of exhaust vents moves in directions that are generally outward from air cleaning apparatus 100, such as shown by directions 530-533. In some embodiments of air cleaning apparatus 100, the average time air spent within the cleaning apparatus without the curved vortex walls is 0.118 seconds, and the average time air spends within air cleaning apparatus 100 when the vortex walls are included within the air cleaning apparatus is 0.173 seconds, which is a 47% gain in the time air spends within the air cleaning apparatus by including the vortex walls.

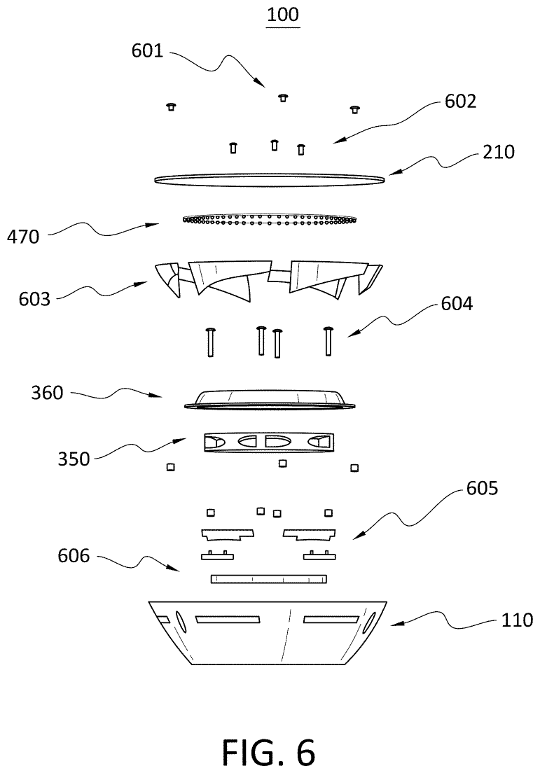

Turning ahead in the drawings, FIG. 6 shows an exploded view of air cleaning apparatus 100. As shown in FIG. 6, air cleaning apparatus can include shell 110, an intake filter 606, such as a 100 PPI (pores per inch) filter, an indicator light 605, fan 350, vortex reflector 360, bolts 604, an exhaust filter 603, such as a 10 PPI filter, LED board 470, back plate 210, press fit fasteners 602, bolts 601, and/or other suitable parts. In some embodiments, an input voltage can include a 12 volt direct current nominal. In various embodiments, the LEDs (e.g., 471, 472) on LED board 470 can a have a power rating of 24 watt and/or a 2 ampere max. In some embodiments, the air cleaning apparatus can be approximately 8 inches in diameter and approximately 2 inches tall (from central surface 111 (FIGS. 1-2) to back plate 210 (FIG. 2)). In many embodiments, shell 110 can be made of polycarbonate plastic or another suitable material. In these or other embodiments, a back plate of the air cleaning apparatus can be made of aluminum or another suitable material. Vortex reflector 360 can be constructed from and/or be coated with polytetrafluoroethylene (PTFE) and/or some other material that is highly UV reflective (e.g., more than 90% reflective of UV light) and/or that diffuses UV light. In this way, UV exposure for air taken into the air cleaning apparatus can be maximized by saturating air chamber 380 (FIG. 3) with UV light. In many embodiments, the LED light can be contained within air cleaning apparatus 100 and not be emitted or broadcast outside the air cleaning apparatus. In a number of embodiments, shielding can be used around the inlet and/or outlet vents to prevent the LED light from being emitted outside such vents.

In these or other embodiments, the air cleaning apparatus can be surface mounted via three #8 mounting screws, such as through screw holes 140 (FIGS. 1-2), and/or using other suitable fasteners, such as screws, nails, hook and loop tape, etc. In some embodiments, air cleaning apparatus 100 can operate in occupied spaces. In many embodiments, air cleaning apparatus 100 can be connected to a 12 volt direct current vehicle ignition wire and/or a manually switched 12 volt direct current wire. Air cleaning apparatus 100 can have an estimated lifespan of approximately 10,000 to approximately 20,000 hours.

In various embodiments, an air flow rate of the air cleaning apparatus can be approximately 10 cubic feet per minute. In some embodiments, an air flow rate of the air cleaning apparatus can be tailored by modulating a voltage supplied to fan. In many embodiments, the air cleaning apparatus can clean approximately 100 cubic feet of air in approximately 10 minutes. In some embodiments, the air cleaning apparatus can disinfect at least 90% of viruses (including but not limited to SARS-CoV-2) and/or bacteria within approximately 30 minutes.

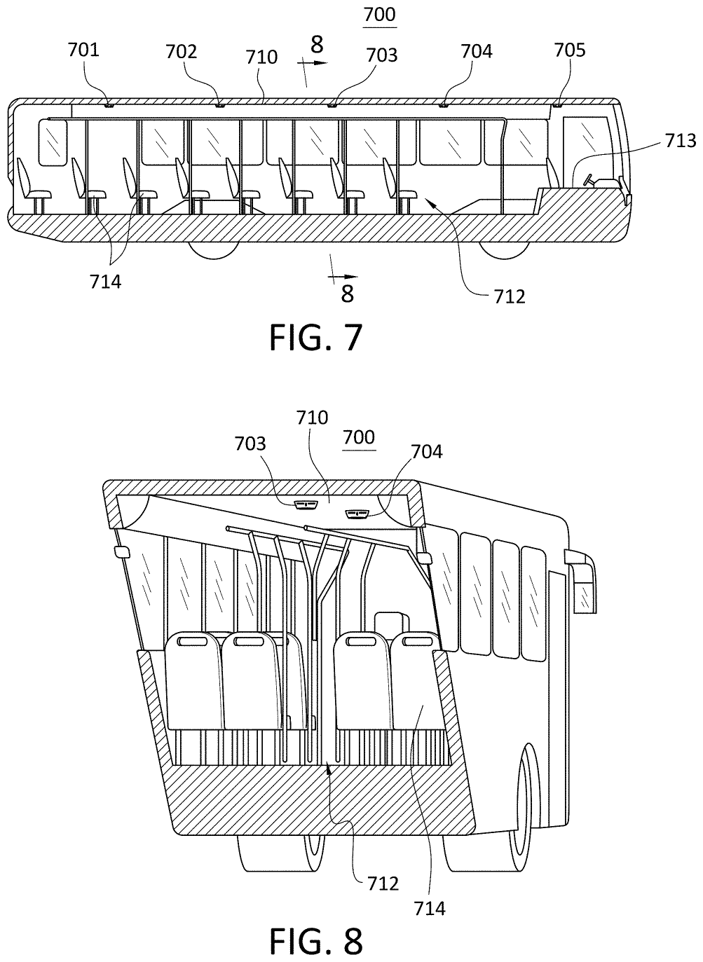

Turning ahead in the drawings, FIG. 7 illustrates a transit bus 700. FIG. 8 illustrates a cross-sectional view, along cross-section line 8-8 in FIG. 7, of transit bus 700. Transit bus 700 is merely exemplary, and embodiments of the transit bus are not limited to the embodiments presented herein. The transit bus can be employed in many different embodiments or examples not specifically depicted or described herein. In many embodiments, transit bus can include air cleaning apparatuses 701-705, which can be similar or identical to air cleaning apparatus 100 (FIGS. 1-6). Air cleaning apparatuses 701-705 can locally disinfect the upper air in transit bus 700. In other embodiments, air cleaning apparatuses (e.g., 100, 701-705) can be used in other transit settings (e.g., subway, trolley, streetcar, airplane, train, etc.).

In some embodiments, air cleaning apparatuses 701-705 can be ceiling mounted (as shown in FIGS. 7-8) on transit bus 700. For example, air cleaning apparatuses 701-705 can be mounted to ceiling 710 of transit bus 700, such as above aisle 712 of bus transit 700 or over driver seat 713 of transit bus 700. In other embodiments, air cleaning apparatuses 701-705 can be mounted to the back of seats 714, wall mounted, etc. In various embodiments, air cleaning apparatuses 701-705 individually can cover 2-3 rows of seating on transit bus 700, or in a car, an airplane, a trolley, or a train. For example, air cleaning apparatuses 701-705 can be mounted every 4 to 6 feet. By using multiple air cleaning apparatuses (e.g., 701-705), the air exhaled by individuals (e.g., the driver and/or passengers) on transit bus 700 (or other suitable transit vehicles) can be cleaned before the air travels far from the respective individuals, which can limit the spread of pathogens. The intake vents (e.g., 120 (FIG. 1)) of air cleaning apparatuses 701-705 are directed downward toward the individuals that use transit bus 700, to pull in the air exhales by the individuals. The exhaust vents (e.g., 130 (FIG. 1)) of air cleaning apparatuses 701-705 are directed outward along ceiling 710, approximately perpendicular to the input direction. In this way, the air cleaning apparatus (e.g., 701-705) will not recycle its own exhaust because the air flow from the intake vent does not mix with the air flow from the outlet vent.

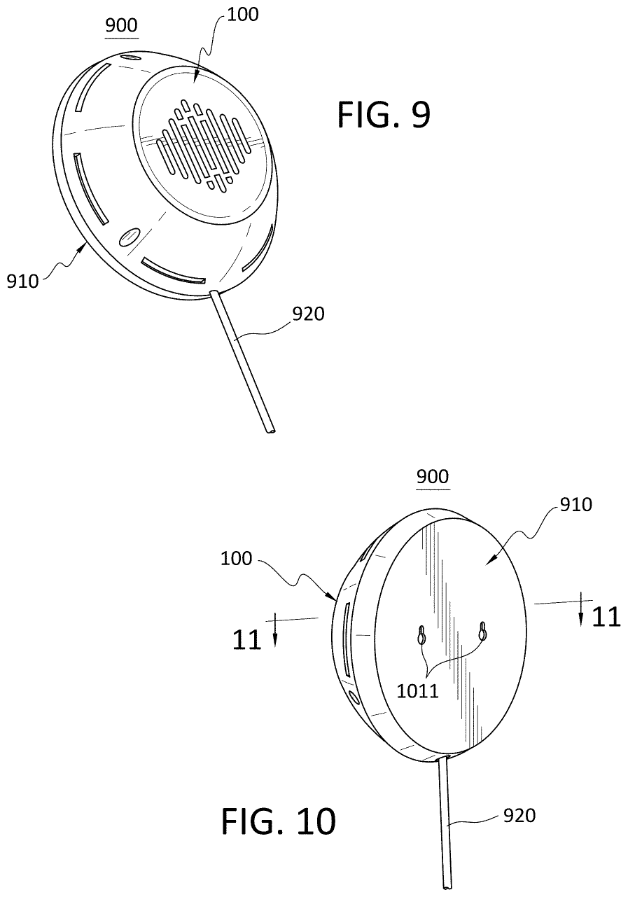

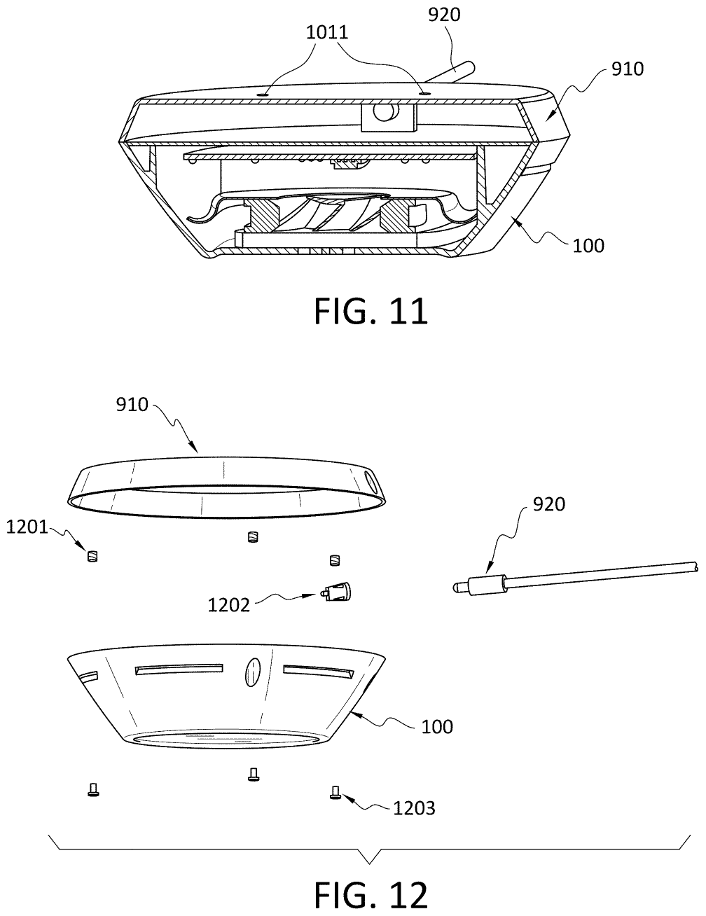

Turning ahead in the drawings, FIG. 9 illustrates a shell-side perspective view of an exemplary embodiment of an air cleaning apparatus 900 with a top hat 910. FIG. 10 illustrates a hat-side perspective view of air cleaning apparatus 900. FIG. 11 illustrates a cross-sectional view, along cross-section line 11-11 in FIG. 10, of air cleaning apparatus 900. FIG. 12 shows an exploded view of air cleaning apparatus 900. Air cleaning apparatus 900 is merely exemplary, and embodiments of the air cleaning apparatus are not limited to the embodiments presented herein. The air cleaning apparatus can be employed in many different embodiments or examples not specifically depicted or described herein.

In many embodiments, air cleaning apparatus 900 can include air cleaning apparatus 100 (FIGS. 1-6), top hat 910, a power cord 920, helical inserts 1201, a power cord socket 1202, and/or bolts 1203. In a number of embodiments, air cleaning apparatus 900 can be used in commercial applications that operate using 120 volt alternating current. A 12 volt direct current power adapter (not shown) can be used, either on the cord (e.g., at a wall wart) or inside top hat 910. Top hat 910 can be used to mount air cleaning apparatus 900 to a wall, such as by using wall mounting holes 1011, or to sit on or be mounted to a table, desk, or other surface. For example, air cleaning apparatus 900 can be used at a cubicle of a worker to disinfect the air exhaled by the worker. In many embodiments, air cleaning apparatus 900 can include a dial/switch (not shown) that can allow a user to vary fan speed/UV output of the apparatus.

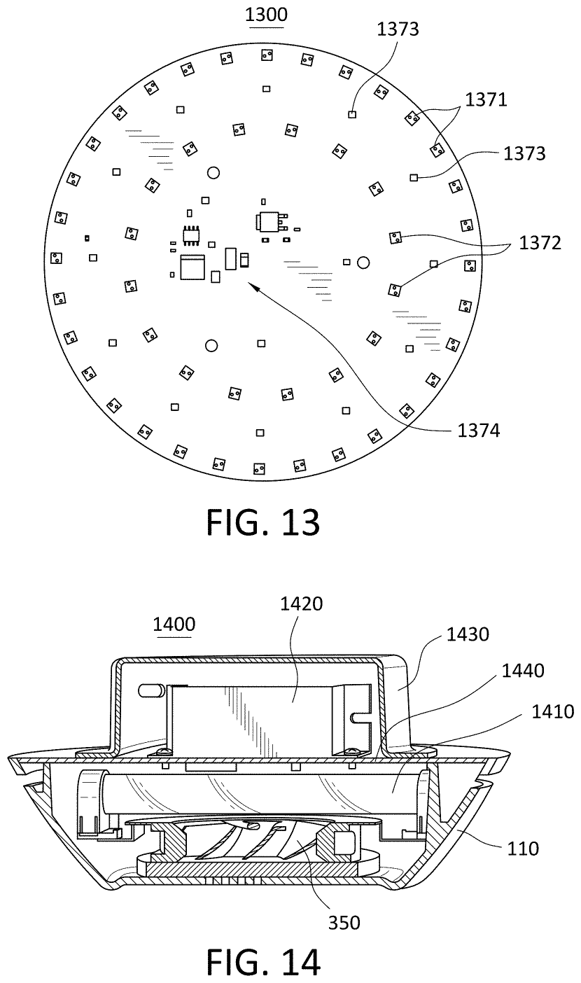

Turning ahead in the drawings, FIG. 13 illustrates an LED board 1300. LED board 1300 is merely exemplary, and embodiments of the LED board are not limited to the embodiments presented herein. The LED board can be employed in many different embodiments or examples not specifically depicted or described herein. LED board 1300 can be similar to LED board 470 (FIGS. 4-6), and various elements of LED board 1300 can be similar or identical to various elements of LED board 470 (FIGS. 4-6). LED board 1300 can be used in air cleaning apparatus 100 (FIGS. 1-6), 701-705 (FIG. 7), and/or 900 (FIGS. 9-12). As shown in FIG. 13, LED board 1300 can include LEDs 1371 in an outer concentric circle, which can be similar or identical to LEDs 471 (FIGS. 4-5), LEDs 1372 in an inner concentric circle, which can be similar or identical to LEDs 472 (FIGS. 4-5), LEDs 1373 in an intermediate concentric circle between the inner concentric circle and the outer concentric circle, and electronic elements 1374 to control LED board 1300. In many embodiments, LEDs 1371 and 1372 and be UV-C LEDs, such as 265 nm LEDs, for example, which can disinfect pathogens. LEDs 1373 can be UV-A LEDs, such as 365 nm LEDs, which can be used to activate a photocatalyst to neutralize volatile organic compounds (VOCs). In a number of embodiments, one or more surfaces inside the air cleaning apparatus (e.g., 100 (FIGS. 1-6), 701-705 (FIG. 7), 900 (FIGS. 9-12)) can be coated with a photocatalyst, such as spraying titanium oxide (TiO₂), on such surfaces. Instead of disinfecting, the UV-A LEDs can activate the photocatalyst to create areas of ionized air that can neutralize VOCs.

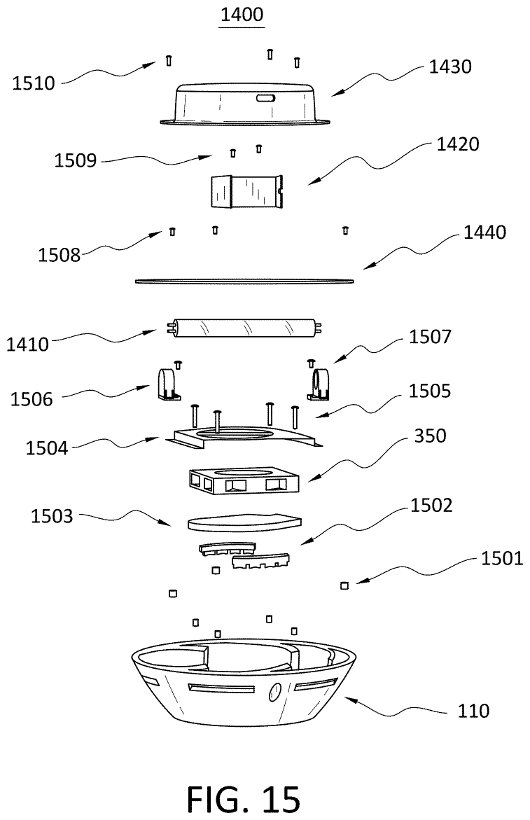

Turning ahead in the drawings, FIG. 14 illustrates a cross-sectional view of an exemplary embodiment of an air cleaning apparatus 1400 with a mercury vapor lamp 1410. FIG. 15 shows an exploded view of air cleaning apparatus 1400. Air cleaning apparatus 1400 is merely exemplary, and embodiments of the air cleaning apparatus are not limited to the embodiments presented herein. The air cleaning apparatus can be employed in many different embodiments or examples not specifically depicted or described herein. Air cleaning apparatus 1400 can be similar to air cleaning apparatus 100 (FIGS. 1-6), 701-705 (FIG. 7), and/or 900 (FIGS. 9-12), and various elements of air cleaning apparatus 1400 can be similar or identical to various elements of air cleaning apparatus 100 (FIGS. 1-6), 701-705 (FIG. 7), and/or 900 (FIGS. 9-12).

In many embodiments, air cleaning apparatus 1400 can include shell 110, helical inserts 1501, an indicator light 1502, an intake filter 1503, such as a 100 PPI (pores per inch) filter, fan 350, a lamp mount plate 1504, bolts 1505, lamp mounts (e.g., T5 tombstone) 1506, bolts 1507, mercury vapor lamp 1410 (e.g., a 4 watt mercury vapor lamp), ballast back plate 1440, bolts 1508, ballast 1420, bolts 1509, ballast top hat 1430, and/or bolts 1510. Air cleaning apparatus 1400 is particularly suitable to be used in fixtures designed for mercury vapor lamps. By contrast, air cleaning apparatus 100 (FIGS. 1-6) has a flat back, less glass, and no mercury, which can be better suited for high vibration environments (e.g., transit applications).

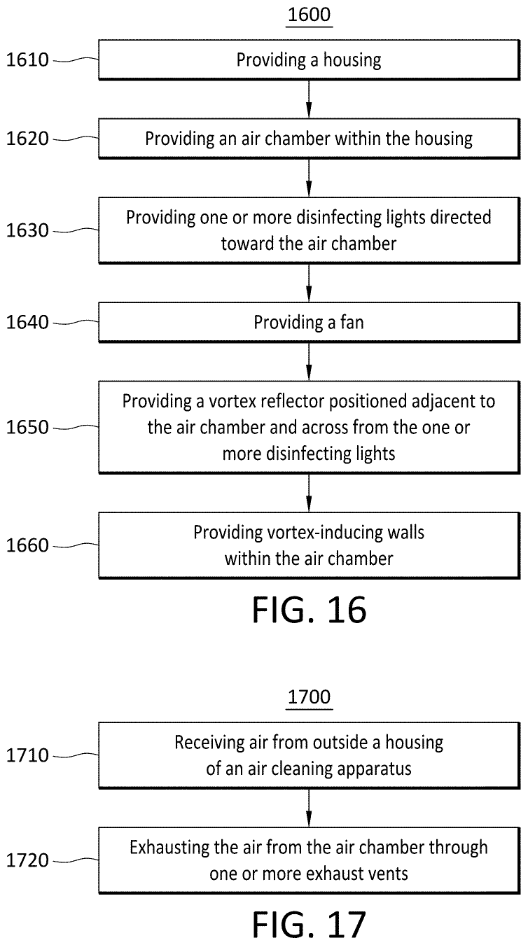

Turning ahead in the drawings, FIG. 16 illustrates a flow chart for a method 1600 of providing an air cleaning apparatus, according to an embodiment. The air cleaning apparatus can be similar or identical to air cleaning apparatus 100 (FIGS. 1-6), 701-705 (FIG. 7), 900 (FIGS. 9-12), and/or 1400 (FIG. 14). Method 1600 is merely exemplary and is not limited to the embodiments presented herein. The method can be employed in many different embodiments or examples not specifically depicted or described herein. In some embodiments, the procedures, the processes, and/or the activities of method 1600 can be performed in the order presented. In other embodiments, the procedures, the processes, and/or the activities of method 1600 can be performed in any suitable order. In still other embodiments, one or more of the procedures, the processes, and/or the activities of method 1600 can be combined or skipped.

Referring to FIG. 16, method 1600 can include an activity 1610 of providing a housing. The housing can be similar or identical to shell 110 (FIG. 1), back plate 210 (FIG. 2), top hat 910 (FIG. 9), and/or ballast top hat 1430. In many embodiments, the housing can include one or more intake vents and one or more exhaust vents. The intake vents can be similar or identical to intake vents 120 (FIG. 1). The exhaust vents can be similar or identical to exhaust vents 130 (FIG. 1).

In a number of embodiments, method 1600 also can include an activity 1620 of providing an air chamber within the housing. The air chamber can be similar or identical to 380 (FIG. 3).

In several of embodiments, method 1600 additionally can include an activity 1630 of providing one or more disinfecting lights directed toward the air chamber. The disinfecting lights can be similar or identical to LEDs 471-472 (FIG. 4), LEDs 1371-1373 (FIG. 13), and/or mercury vapor lamp 1410 (FIG. 14).

In a number of embodiments, method 1600 further can include an activity 1640 of providing a fan. The fan can be similar or identical to fan 350 (FIG. 3, 14). In many embodiments, the fan can be operable to perform moving air from outside the housing in a first direction through the one or more intake vents and into the air chamber to be exposed to the one or more disinfecting lights. The first direction can be similar or identical to direction 520 (FIG. 5). After the air is exposed within the air chamber to the one or more disinfecting lights, the fan also can be operable to perform moving the air from of the air chamber through the one or more exhaust vents in one or more second directions that are other than approximately parallel to the first direction. The second directions can be similar or identical to directions 530-533 (FIG. 5).

In several embodiments, method 1600 further can include an activity 1650 of providing a vortex reflector positioned adjacent to the air chamber and across from the one or more disinfecting lights. The vortex reflector can be similar or identical to vortex reflector 360 (FIG. 3).

In a number of embodiments, method 1600 further can include an activity 1660 of providing vortex-inducing walls within the air chamber. The vortex-inducing walls can be similar or identical to vortex inducing walls 370 (FIG. 3).

Turning ahead in the drawings, FIG. 17 illustrates a flow chart for a method 1700 of cleaning air, according to an embodiment. Method 1700 is merely exemplary and is not limited to the embodiments presented herein. The method can be employed in many different embodiments or examples not specifically depicted or described herein. In some embodiments, the procedures, the processes, and/or the activities of method 1700 can be performed in the order presented. In other embodiments, the procedures, the processes, and/or the activities of method 1700 can be performed in any suitable order. In still other embodiments, one or more of the procedures, the processes, and/or the activities of method 1700 can be combined or skipped.

Referring to FIG. 17, method 1700 can include an activity 1710 of receiving air from outside a housing of an air cleaning apparatus in a first direction through one or more intake vents of the housing and into an air chamber of the air cleaning apparatus to be exposed to one or more disinfecting lights of the air cleaning apparatus that are directed toward the air chamber. The housing can be similar or identical to shell 110 (FIG. 1), back plate 210 (FIG. 2), top hat 910 (FIG. 9), and/or ballast top hat 1430. The air cleaning apparatus can be similar or identical to air cleaning apparatus 100 (FIGS. 1-6), 701-705 (FIG. 7), 900 (FIGS. 9-12), and/or 1400 (FIG. 14). The first direction can be similar or identical to direction 520 (FIG. 5). The intake vents can be similar or identical to intake vents 120 (FIG. 1). The air chamber can be similar or identical to 380 (FIG. 3). The disinfecting lights can be similar or identical to LEDs 471-472 (FIG. 4), LEDs 1371-1373 (FIG. 13), and/or mercury vapor lamp 1410 (FIG. 14). In a number of embodiments, the air cleaning apparatus can include a vortex reflector positioned adjacent to the air chamber and across from the one or more disinfecting lights. The vortex reflector can be similar or identical to vortex reflector 360 (FIG. 3). In various embodiments, the air cleaning apparatus can include vortex-inducing walls within the air chamber. The vortex-inducing walls can be similar or identical to vortex inducing walls 370 (FIG. 3)

In a number of embodiments, method 1700 also can include, after the air is exposed within the air chamber to the one or more disinfecting lights, an activity 1720 of exhausting the air from of the air chamber through one or more exhaust vents of the housing in one or more second directions that are other than approximately parallel to the first direction. The exhaust vents can be similar or identical to exhaust vents 130 (FIG. 1). The second directions can be similar or identical to directions 530-533 (FIG. 5).

The foregoing is provided for purposes of illustrating, explaining, and describing embodiments of these disclosures. Modifications and adaptations to these embodiments will be apparent to those skilled in the art and may be made without departing from the scope or spirit of these disclosures.

Although the air cleaning apparatus has been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-17 may be modified, and that the foregoing discussion of certain of these embodiments does not necessarily represent a complete description of all possible embodiments. For example, one or more of the procedures, processes, or activities of FIG. 16-17 may include different procedures, processes, and/or activities and be performed by many different modules, in many different orders, and/or one or more of the procedures, processes, or activities of FIGS. 16-17 may include one or more of the procedures, processes, or activities of another different one of FIGS. 16-17. As another example, the elements within air cleaning apparatus 100 (FIGS. 1-6), 701-705 (FIG. 7), 900 (FIGS. 9-12), and/or 1400 (FIG. 14) can be interchanged or otherwise modified.

Replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. An air cleaning apparatus comprising:
a housing comprising:
one or more intake vents; and
one or more exhaust vents;
an air chamber within the housing;
one or more disinfecting lights directed toward the air chamber;
a fan operable to perform:
moving air from outside the housing in a first direction through the one or more intake vents and into the air chamber to be exposed to the one or more disinfecting lights; and
after the air is exposed within the air chamber to the one or more disinfecting lights, moving the air from of the air chamber through the one or more exhaust vents in one or more second directions that are other than approximately parallel to the first direction;
a vortex reflector positioned adjacent to the air chamber and across from the one or more disinfecting lights; and
vortex-inducing walls within the air chamber.

2. The air cleaning apparatus of claim 1, wherein the one or more second directions are approximately perpendicular to the first direction.

3. The air cleaning apparatus of claim 1, wherein:
the one or more intake vents are located proximate a center of the housing; and
the one or more exhaust vents are located proximate an outer edge of the housing.

4. The air cleaning apparatus of claim 1, wherein the one or more disinfecting lights comprises UV-C LEDs.

5. The air cleaning apparatus of claim 4, wherein the UV-C LEDs are arranged in concentric circles within the housing.

6. The air cleaning apparatus of claim 1, wherein:
the one or more disinfecting lights comprises a mercury vapor design.

7. The air cleaning apparatus of claim 1, wherein the one or more disinfecting lights comprises a mercury vapor design.

8. The air cleaning apparatus of claim 1, wherein the vortex reflector comprises a material that reflects more than 90% of ultraviolet light generated by the one or more disinfecting lights and diffuses the ultraviolet light within the air chamber.

9. The air cleaning apparatus of claim 1, wherein the vortex reflector comprises a concave shape relative to the air chamber to facilitate redirecting the air that is moving radially outward to circulate back inward within the air chamber.

10. The air cleaning apparatus of claim 1, wherein the vortex-inducing walls curve radially inward across the one or more exhaust vents.

11. The air cleaning apparatus of claim 1, wherein the vortex-inducing walls facilitate redirecting the air that is moving radially outward to a circular direction around a perimeter of the air chamber.

12. The air cleaning apparatus of claim 1, wherein the fan is proximate the one or more intake vents and is operable to cool the one or more disinfecting lights.

13. The air cleaning apparatus of claim 1, wherein an inner surface of the housing comprises a photocatalyst to neutralize volatile organic compounds.

14. The air cleaning apparatus of claim 13 further comprising one or more UV-A lights operable to activate the photocatalyst.

15. A transit vehicle comprising one or more of the air cleaning apparatus of claim 1, wherein the one or more of the air cleaning apparatus are located on a ceiling of the transit vehicle.

16. The transit vehicle of claim 15, wherein the one or more of the air cleaning apparatus are oriented with the one or more intake vents directed approximately away from the ceiling and the one or more exhaust vents directed approximately parallel to the ceiling:

17. The transit vehicle of claim 15, wherein the one or more of the air cleaning apparatus comprise at least five of the air cleaning apparatus of claim 1.

18. The transit vehicle of claim 15, wherein the at least five of the air cleaning apparatus are spaced at least approximately 4 feet apart from each other.

19. A method of providing an air cleaning apparatus, comprising:
providing a housing comprising:
one or more intake vents; and
one or more exhaust vents;
providing an air chamber within the housing;
providing one or more disinfecting lights directed toward the air chamber;
providing a fan operable to perform:
moving air from outside the housing in a first direction through the one or more intake vents and into the air chamber to be exposed to the one or more disinfecting lights; and
after the air is exposed within the air chamber to the one or more disinfecting lights, moving the air from of the air chamber through the one or more exhaust vents in one or more second directions that are other than approximately parallel to the first direction;
providing a vortex reflector positioned adjacent to the air chamber and across from the one or more disinfecting lights; and
providing vortex-inducing walls within the air chamber.

20. A method of cleaning air comprising:
receiving air from outside a housing of an air cleaning apparatus in a first direction through one or more intake vents of the housing and into an air chamber of the air cleaning apparatus to be exposed to one or more disinfecting lights of the air cleaning apparatus that are directed toward the air chamber, wherein the air cleaning apparatus comprises a vortex reflector positioned adjacent to the air chamber and across from the one or more disinfecting lights, and the air cleaning apparatus comprises vortex-inducing walls within the air chamber; and
after the air is exposed within the air chamber to the one or more disinfecting lights, exhausting the air from of the air chamber through one or more exhaust vents of the housing in one or more second directions that are other than approximately parallel to the first direction.
